(19) United States
(12) Patent Application Publication
(10) Pub. No.: US 2016/0345930 A1
(43) Pub. Date: Dec. 1, 2016

(54) BLOOD PRESSURE MEASUREMENT DEVICE AND BLOOD PRESSURE MEASUREMENT METHOD

(71) Applicants: SEIKO EPSON CORPORATION, Tokyo (JP); NATIONAL CEREBRAL AND CARDIOVASCULAR CENTER, Suita-shi (JP)

(72) Inventors: Hiromitsu MIZUKAMI, Matsumoto-shi (JP); Kazunori UEMURA, Ibaraki-shi (JP); Masaru SUGIMACHI, Toyonaka-shi (JP)

(73) Assignees: SEIKO EPSON CORPORATION, Tokyo (JP); NATIONAL CEREBRAL AND CARDIOVASCULAR CENTER, Suita-shi (JP)

(21) Appl. No.: 15/155,584

(22) Filed: May 16, 2016

(30) Foreign Application Priority Data
May 25, 2015 (JP) 2015-105496

(51) Int. Cl. A61B 8/04; A61B 8/08; A61B 8/02; A61B 8/00

(52) U.S. Cl. CPC A61B 8/04; A61B 8/4444; A61B 8/5207; A61B 8/02; A61B 8/58

(57) ABSTRACT

A first blood vessel diameter and a second blood vessel diameter are measured using a first ultrasonic probe and a second ultrasonic probe that are provided close to a blood vessel of a subject so as to be situated at a given distance (Lp). Characteristic phases of a pulse wave are determined from the peak of a second-order differential value of the first blood vessel diameter and the peak of a second-order differential value of the second blood vessel diameter, and the difference (Δt) in pulse wave transit time is calculated from the difference in timing between the characteristic phases to calculate pulse wave velocity (PWV). A given calculation process that uses the pulse wave velocity (PWV) and the measured blood vessel diameter as variables is performed to calculate blood pressure.

Japanese Patent Application No. 2015-105496 filed on May 25, 2015, is hereby incorporated by reference in its entirety.

### BACKGROUND

A non-invasive blood pressure measurement method that utilizes an ultrasonic wave is known as a blood pressure measurement method that does not utilize pressure. For example, JP-A-2004-41382 discloses a method that calculates blood pressure from a stiffness parameter β (that represents the stiffness of a blood vessel) and a blood vessel diameter on the assumption that a change in blood pressure and a blood vessel diameter have a non-linear relationship.

It may be desired to accurately measure blood pressure even when using a non-invasive blood pressure measurement method, and implement continuous blood pressure measurement on a beat basis. When calculating blood pressure based on the blood vessel diameter, it is necessary to maintain the blood vessel diameter measurement accuracy as high as about several tens of nanometers to several micrometers in order to meet the required blood pressure measurement accuracy. It is relatively easy to implement such high-accuracy blood vessel diameter measurement when the subject is in a resting state (i.e., within a short time), but it may be difficult to continuously implement high-accuracy blood vessel diameter measurement. This is because the subject may make a body motion during continuous measurement. Specifically, the blood vessel may expand and contract due to a body motion of the subject, or the relative positions of the measurement device and the blood vessel may change due to a body motion of the subject. In such a case, it is necessary to calculate blood pressure taking account of a situation in which an error may be included in the measured blood vessel diameter.

The method disclosed in JP-A-2004-41382 (hereinafter referred to as “known technology”) calculates blood pressure P from a blood vessel diameter D based on the following equations (1) and (2). According to this method, since the measured blood vessel diameter D serves as an exponent, a small measurement error in the blood vessel diameter D significantly affects the blood pressure P to be measured. Therefore, it is considered that the known technology is not suitable for continuous measurement.

P(D)=Pd·exp[β·(D/Dd−1)] (1)

β=ln(Ps/Pd)/(Ds/Dd−1) (2)

where, P is the measured blood pressure, Ps is the systolic blood pressure, Pd is the diastolic blood pressure, D is the measured blood vessel diameter, Ds is the systolic blood vessel diameter, and Dd is the diastolic blood vessel diameter.

### SUMMARY

According to one aspect of the invention, there is provided a blood pressure measurement device comprising: a blood vessel diameter measurement section that measures a blood vessel diameter of an artery; a pulse wave velocity measurement section that measures a pulse wave velocity through the artery; and a blood pressure calculation section that performs a given calculation process that uses the blood vessel diameter and the pulse wave velocity as variables to calculate blood pressure.

According to another aspect of the invention, there is provided a blood pressure measurement method comprising: measuring a blood vessel diameter of an artery; measuring a pulse wave velocity through the artery; and performing a given calculation process that uses the blood vessel diameter and the pulse wave velocity as variables to calculate blood pressure.

### BRIEF DESCRIPTION OF THE DRAWINGS

FIG. 1 is a view illustrating a system configuration example of a blood pressure measurement device.

FIG. 2 is a view illustrating the attachment state of a first ultrasonic probe and a second ultrasonic probe.

FIG. 3 is a cross-sectional view illustrating the attachment position of a first ultrasonic probe and a second ultrasonic probe.

FIG. 4A is a view illustrating an example of time-series waveforms of a first blood vessel diameter D1 and a second blood vessel diameter D2.

FIG. 4B is a view illustrating an acceleration waveform obtained by subjecting each time-series waveform illustrated in FIG. 4A to second-order differentiation.

FIG. 4C is an enlarged view illustrating part of each acceleration waveform illustrated in FIG. 4B.

FIG. 5 is a graph illustrating an example of relation between blood vessel diameter and blood pressure in an unpressurized state.

FIG. 6 is a block diagram illustrating a functional configuration example of a blood pressure measurement device.

FIG. 7 is a view illustrating a data configuration example of blood vessel diameter log data.

FIG. 8 is a view illustrating a data configuration example of blood pressure log data.

FIG. 9 is a flowchart illustrating the flow of the main process performed by a blood pressure measurement device.

FIG. 10 is a flowchart illustrating the flow of a calibration process.

FIG. 11 is a flowchart illustrating the flow of a pulse wave velocity measurement process.

FIG. 12 is a flowchart illustrating the flow of a blood pressure calculation process.

FIG. 13 is a graph illustrating the results of blood pressure measurement that uses a known β method (obtained by a validation test).

FIG. 14 is a graph illustrating the results of blood pressure measurement that uses a PWV method (obtained by a validation test).

FIG. 15 is a flowchart illustrating the flow of a calibration process according to a modification.

FIG. 16 is a flowchart illustrating the flow of a blood pressure calculation process according to a first modification.

FIG. 17 is a flowchart illustrating the flow of a blood pressure calculation process according to a second modification.

### DESCRIPTION OF EXEMPLARY EMBODIMENTS

Several embodiments of the invention may provide technology that implements non-invasive blood pressure measurement that exhibits excellent robustness with respect to a body motion of the subject, and makes it possible to implement high-accuracy continuous blood pressure measurement.

According to one embodiment of the invention, there is provided a blood pressure measurement device comprising: a blood vessel diameter measurement section that measures a blood vessel diameter of an artery; a pulse wave velocity measurement section that measures a pulse wave velocity through the artery; and a blood pressure calculation section that performs a given calculation process that uses the blood vessel diameter and the pulse wave velocity as variables to calculate blood pressure.

In the blood pressure measurement device, the blood pressure calculation section may calculate the blood pressure by performing the calculation process in which the blood pressure is proportional to a second power of the pulse wave velocity, and is proportional to a reciprocal of the blood vessel diameter.

According to this configuration, the blood pressure can be calculated by performing the calculation process that uses the non-invasively measured blood vessel diameter and the pulse wave velocity as variables. Since the pulse wave velocity is measured from the timing of the change point of the blood vessel diameter, the measurement of the pulse wave velocity is not easily affected by a body motion of the subject. This makes it possible to implement non-invasive blood pressure measurement that exhibits excellent robustness with respect to a body motion of the subject as compared with the known blood pressure calculation process that uses only the blood vessel diameter, and implement high-accuracy continuous blood pressure measurement.

In the blood pressure measurement device, the blood vessel diameter measurement section and the pulse wave velocity measurement section may perform measurement with respect to an identical characteristic phase of a pulse wave.

According to this configuration, since the blood vessel diameter and the pulse wave velocity are measured with respect to an identical characteristic phase, it is possible to maintain the blood pressure measurement (calculation) accuracy at a high level.

The blood pressure measurement device may further comprise: a characteristic phase determination section that performs a given differential calculation process on a waveform that represents a temporal change in the blood vessel diameter to determine a diastolic phase and a notch phase of a pulse wave.

The characteristic phase of the pulse wave can be determined from the waveform that represents a temporal change in blood vessel diameter. However, the waveform that represents a temporal change in blood vessel diameter is not necessarily a waveform from which the characteristic phase can be easily detected. It is possible to easily detect the change point of the waveform, and clearly detect the characteristic phase by performing the differential calculation process on the waveform that represents a temporal change in blood vessel diameter. This makes it possible to improve the characteristic phase detection accuracy and the blood pressure measurement accuracy.

In the blood pressure measurement device, the blood vessel diameter measurement section may measure a diastolic blood vessel diameter and a notch blood vessel diameter, the pulse wave velocity measurement section may measure a diastolic pulse wave velocity and a notch pulse wave velocity, and the blood pressure calculation section may calculate diastolic blood pressure by performing the calculation process that uses the diastolic blood vessel diameter and the diastolic pulse wave velocity, calculating notch blood pressure by performing the calculation process that uses the notch blood vessel diameter and the notch pulse wave velocity, and calculating systolic blood pressure by performing a given systolic blood pressure estimation-calculation process that uses the diastolic blood pressure and the notch blood pressure.

According to this configuration, since the systolic blood pressure is calculated from the diastolic blood pressure and the notch blood pressure that have been measured with high accuracy, it is possible to obtain high measurement accuracy with regard to the systolic blood pressure.

In the blood pressure measurement device, the blood pressure calculation section may calculate the systolic blood pressure by performing the systolic blood pressure estimation-calculation process on an assumption that the notch blood pressure is an average arterial pressure.

The blood pressure measurement device may further comprise: ultrasonic probes that transmit and receive an ultrasonic wave to and from the artery, the ultrasonic probes including a first ultrasonic probe that is in charge of an upstream side of the artery, and a second ultrasonic probe that is in charge of a downstream side of the artery, the blood vessel diameter measurement section measuring the blood vessel diameter based on a reception signal received by the first ultrasonic probe or a reception signal received by the second ultrasonic probe, and the pulse wave velocity measurement section measuring the pulse wave velocity based on the reception signal received by the first ultrasonic probe and the reception signal received by the second ultrasonic probe.

According to this configuration, it is possible to measure the pulse wave velocity using two ultrasonic probes.

In the blood pressure measurement device, the first ultrasonic probe and the second ultrasonic probe may transmit and receive the ultrasonic wave to and from the carotid artery, subclavian artery, or aorta.

According to this configuration, since an artery that changes in blood vessel diameter to a relatively small extent due to the effect of sympathetic tone is used as the measurement target, it is possible to obtain stable blood pressure measurement accuracy.

In the blood pressure measurement device, the blood vessel diameter measurement section may measure the blood vessel diameter based on the reception signal received by the first ultrasonic probe and the blood vessel diameter based on the reception signal received by the second ultrasonic probe, and the blood pressure calculation section may use the blood vessel diameter based on the reception signal received by the first ultrasonic probe or the blood vessel diameter based on the reception signal received by the second ultrasonic probe, whichever is larger with respect to a change due to pulsation.

When measuring a change in blood vessel diameter due to pulsation by applying an ultrasonic wave, a change in blood vessel diameter becomes a maximum when the application (irradiation) direction coincides with the diameter of the cross section of the blood vessel in the minor-axis direction. The blood vessel diameter can be measured with higher accuracy as a change in blood vessel diameter that can be measured increases. Therefore, it is possible to further improve the blood vessel diameter measurement accuracy.

In the blood pressure measurement device, the first ultrasonic probe and the second ultrasonic probe may be small-sized probes that are attached to a skin surface of a subject.

According to this configuration, since the position of the ultrasonic probe does not easily change, it is possible to implement high-accuracy continuous measurement. Since the burden imposed on the subject can be reduced as compared with a method that secures the blood pressure measurement device using a belt or the like, this configuration is preferable for continuous long-time measurement. It is also possible to improve workability when attaching the blood pressure measurement device.

According to another embodiment of the invention, there is provided a blood pressure measurement method comprising: measuring a blood vessel diameter of an artery; measuring a pulse wave velocity through the artery; and performing a given calculation process that uses the blood vessel diameter and the pulse wave velocity as variables to calculate blood pressure.

### First Embodiment

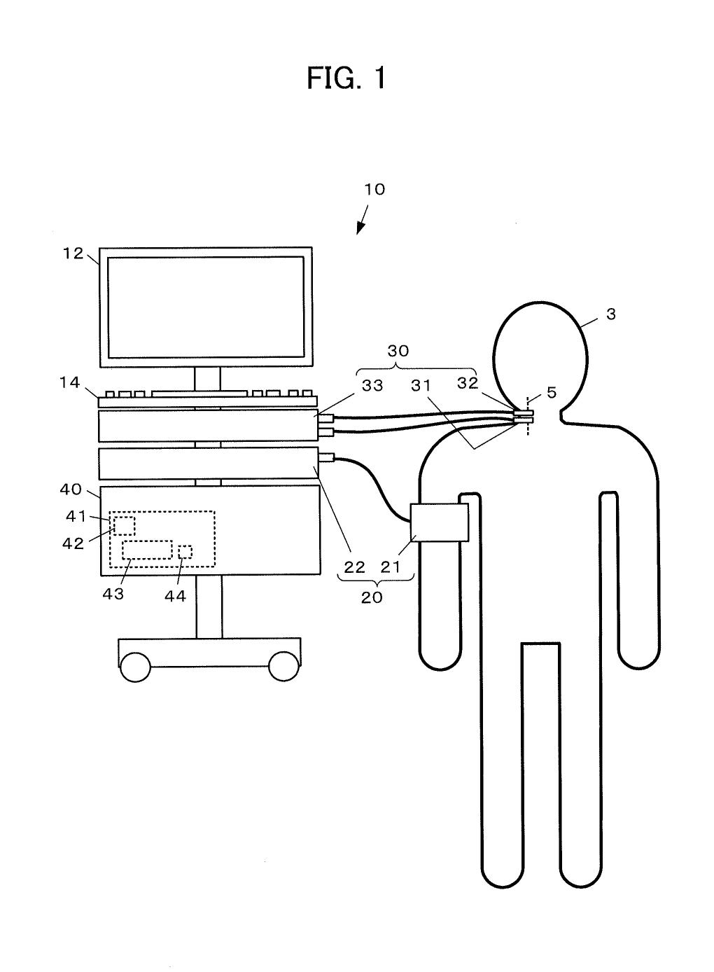

FIG. 1 is a view illustrating a system configuration example of a blood pressure measurement device 10 according to a first embodiment. The blood pressure measurement device 10 is a device that measures the blood pressure by non-invasively measuring the diameter of a blood vessel 5 of a subject 3 and the pulse wave velocity through ultrasonic measurement.

The blood pressure measurement device 10 according to the first embodiment includes 1) a touch panel 12 that serves as a means that displays the measurement results and operation information as an image, and an operation input means, 2) a keyboard 14 that is used to perform an operation input, 3) a calibration blood pressure measurement unit 20, 4) an ultrasonic measurement control unit 30, and 5) a processing device 40.

The blood pressure measurement device 10 appropriately further includes a power supply and the like (not illustrated in FIG. 1).

The calibration blood pressure measurement unit 20 is a device that measures the blood pressure necessary for calibration for measuring the blood vessel diameter. In the first embodiment, the calibration blood pressure measurement unit 20 may be implemented using an inflation-type sphygmomanometer (cuff-type sphygmomanometer) that includes a cuff 21, and a main device 22 that calculates the blood pressure, and outputs the measured value to the processing device 40. Note that the calibration blood pressure measurement unit 20 may be implemented using a sphygmomanometer other than an inflation-type sphygmomanometer.

The ultrasonic measurement control unit 30 includes a first ultrasonic probe 31, a second ultrasonic probe 32, and a control device 33.

The first ultrasonic probe 31 and the second ultrasonic probe 32 are thin or sheet-like probes that are attached to the skin of the subject 3. The first ultrasonic probe 31 and the second ultrasonic probe 32 are attached so as to be situated at a probe-to-probe distance Lp and measure the minor-axis cross section of the identical blood vessel 5. The first ultrasonic probe 31 and the second ultrasonic probe 32 apply an ultrasonic pulse to the subject 3, and receive the reflected wave.

The control device 33 is a device that controls the first ultrasonic probe 31 and the second ultrasonic probe 32. The control device 33 controls the transmission and the reception of an ultrasonic wave, and outputs signals (reception signals) of the reflected wave received by the first ultrasonic probe 31 and the second ultrasonic probe 32 to the processing device 40.

The processing device 40 is a key device of the blood pressure measurement device 10. The processing device 40 is connected to each section (e.g., touch panel 12, keyboard 14, calibration blood pressure measurement unit 20, and ultrasonic measurement control unit 30) of the blood pressure measurement device 10 so as to be able to exchange signals with each section. The processing device 40 may include a communication device that communicates with an external device.

The processing device 40 includes a control board 41. The control board 41 is provided with a central processing unit (CPU) 42, a storage medium 43 (e.g., integrated circuit (IC) memory and hard disk), and a communication IC 44 that implements data communication with the calibration blood pressure measurement unit 20 and the ultrasonic measurement control unit 30. The CPU 42 controls the blood pressure measurement device 10 by executing a program stored in the storage medium 43, and implements various functions such as a blood pressure measurement function and a measurement result display-storage function. The functions implemented by the CPU 42 may include a function that displays the blood pressure measured by the calibration blood pressure measurement unit 20 on the touch panel 12, and a function that displays raw data that represents the reception signal from the ultrasonic measurement control unit 30 and data that represents a reflected ultrasonic wave signal (e.g., A-mode, B-mode, or M-mode) on the touch panel 12.

Although FIG. 1 illustrates an example in which the calibration blood pressure measurement unit 20 and the ultrasonic measurement control unit 30 are integrally provided to the blood pressure measurement device 10, the configuration is not limited thereto. For example, either or both of the calibration blood pressure measurement unit 20 and the ultrasonic measurement control unit 30 may be provided separately from the blood pressure measurement device 10, and connected to the processing device 40 through a cable or wireless communication channel so as to be able to implement data communication with the processing device 40.

Measurement Principle

The principle of the blood pressure measurement according to the first embodiment is described below.

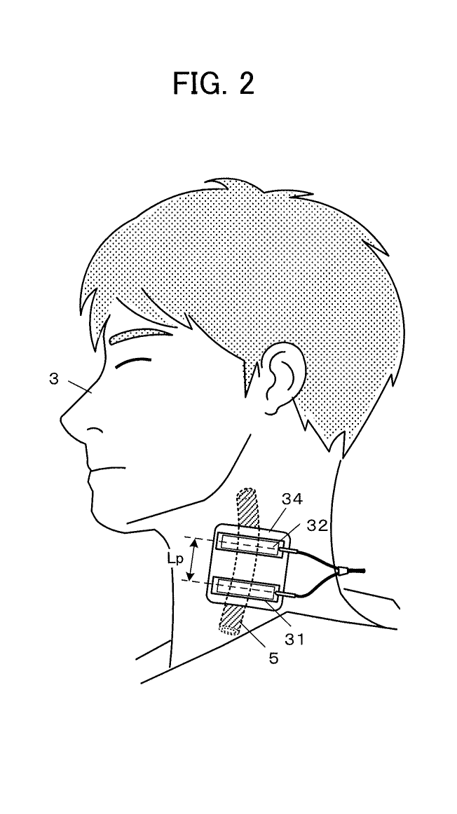

FIG. 2 is a view illustrating the attachment state of the first ultrasonic probe 31 and the second ultrasonic probe 32. The first ultrasonic probe 31 and the second ultrasonic probe 32 are ultrasonic probes that are produced in accordance with identical specifications. The first ultrasonic probe 31 and the second ultrasonic probe 32 are secured on an adhesive base 34 so as to be situated at a given probe-to-probe distance Lp (preferably about 10 mm to about 30 mm) so that the scanning planes thereof are parallel to each other. The adhesive base 34 includes a pressure-sensitive adhesive layer that can be removably attached to the surface of skin. The adhesive base 34 is not easily separated or removed even if the subject 3 makes a body motion. The adhesive base 34 is attached so that the first ultrasonic probe 31 and the second ultrasonic probe 32 can visualize the minor axis of the blood vessel 5 (carotid artery in the first embodiment), the first ultrasonic probe 31 is situated on the side of the heart (upstream side), and the second ultrasonic probe 32 is situated on the side of the head (downstream side).

Note that the first ultrasonic probe 31 and the second ultrasonic probe 32 may be provided to different adhesive bases 34 instead of providing the first ultrasonic probe 31 and the second ultrasonic probe 32 to an identical adhesive base 34.

The measurement target blood vessel 5 is not limited to the carotid artery, but may be another artery that changes in blood vessel diameter to a relatively small extent due to the effect of sympathetic tone, such as the subclavian artery or the aorta.

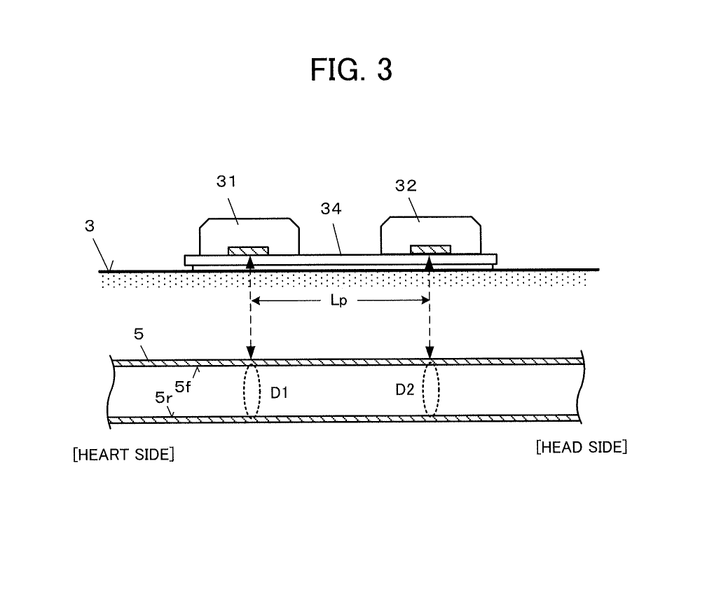

FIG. 3 is a cross-sectional view illustrating the attachment position of the first ultrasonic probe 31 and the second ultrasonic probe 32. The first ultrasonic probe 31 and the second ultrasonic probe 32 transmit an ultrasonic pulse signal or a burst signal having a frequency of several to several tens of MHz from a built-in transmitter toward the blood vessel 5, and receive the reflected wave from a front wall 5f of the blood vessel 5 and the reflected wave from a rear wall 5r of the blood vessel 5 using a built-in receiver. The processing device 40 calculates the diameter of the blood vessel 5 (i.e., a first blood vessel diameter D1 measured by the first ultrasonic probe 31, and a second blood vessel diameter D2 measured by the second ultrasonic probe 32) from the time difference of arrival between the wave received from the front wall 5f and the wave received from the rear wall 5r. The transmission of the ultrasonic wave and the reception of the reflected wave are successively performed at a very short time interval. Therefore, it is possible to successively calculate the first blood vessel diameter D1 and the second blood vessel diameter D2. A waveform in which the blood vessel diameter changes in time series can thus be obtained.

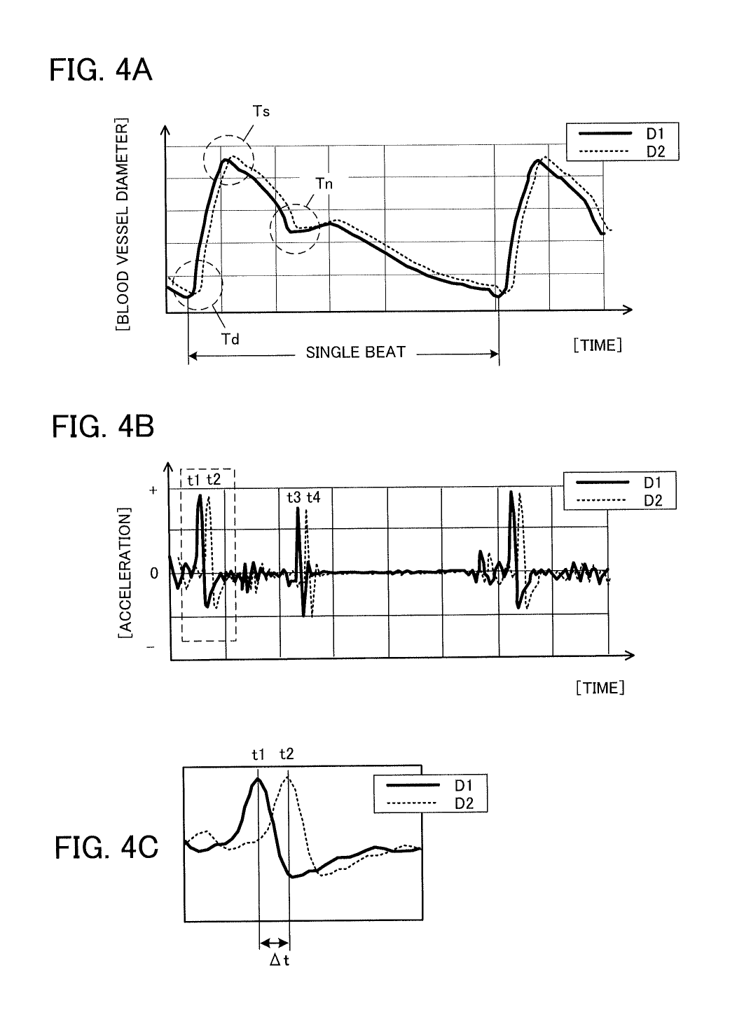

FIG. 4A is a view illustrating an example of time-series waveforms of the first blood vessel diameter D1 and the second blood vessel diameter D2. FIG. 4B is a view illustrating an acceleration waveform obtained by subjecting each time-series waveform illustrated in FIG. 4A to second-order differentiation. FIG. 4C is an enlarged view illustrating part of each acceleration waveform illustrated in FIG. 4B that is enclosed by the broken line that draws a rectangle. Note that each waveform is drawn in a simplified manner in order to facilitate understanding.

As illustrated in FIG. 4A, a diastolic phase Td, a systolic phase Ts, and a notch phase Tn can be determined from the change in the first blood vessel diameter D1 and the change in the second blood vessel diameter D2. Since the first ultrasonic probe 31 is situated closer to the heart than the second ultrasonic probe 32, the systolic pressure wave reaches the first ultrasonic probe 31 at a timing earlier than the timing at which the systolic pressure wave reaches the second ultrasonic probe 32. Therefore, the diastolic/systolic phase observed from the first blood vessel diameter D1 occurs at a timing earlier than that observed from the second blood vessel diameter D2.

However, the diastolic phase Td, the systolic phase Ts, and the notch phase Tn are not necessarily clearly observed from a change in blood vessel diameter, differing from the example illustrated in FIG. 4A. In particular, it is relatively difficult to clearly determine (detect) the peak of the systolic phase Ts (e.g., due to the effect of a cardiac murmur when the subject 3 has cardiac disease or the like).

In the first embodiment, the peak of the diastolic phase Td and the peak of the notch phase Tn are detected instead of detecting the peak of the systolic phase Ts in order to deal with the above problem. More specifically, the first blood vessel diameter D1 and the second blood vessel diameter D2 are successively subjected to second-order differentiation at a time t to calculate the acceleration of the change in diameter. The diastolic phase Td and the notch phase Tn are detected by finding a peak at which the second-order differential value satisfies a given peak condition (e.g., a condition whereby the second-order differential value exceeds a reference value). According to this method, it is possible to reliably detect (find) the diastolic phase Td and the notch phase Tn. Note that second-order differentiation is an example of a given differential calculation process.

Note that the use of the second-order differential value additionally improves the robustness of the blood vessel diameter measurement. Specifically, when the direction of the ultrasonic wave transmitted from the first ultrasonic probe 31 or the second ultrasonic probe 32 (hereinafter referred to as “transmission line”) passes through the center of the cross section of the blood vessel 5 in the minor-axis direction, a change in blood vessel diameter that appears on the transmission line becomes a maximum, and is clearly observed from the waveform. However, when the transmission line does not pass through the center of the cross section of the blood vessel 5 in the minor-axis direction, a change in blood vessel diameter decreases, and the waveform is rounded. When using a configuration that finds the diastolic phase Td and the notch phase Tn from the peak of the blood vessel diameter waveform without performing a differential calculation process, the transmission line may be shifted with respect to the blood vessel 5 due to a body motion of the subject 3, and it may be difficult to find the diastolic phase Td and the notch phase Tn (i.e., it may be difficult to implement continuous measurement) since the peak of the blood vessel diameter waveform may not be observed. When second-order differentiation is used as described above, a clear peak is observed from the acceleration waveform even if the transmission line does not pass through the center of the cross section of the blood vessel 5 in the minor-axis direction as long as the wall of the blood vessel 5 is determined. Specifically, it is possible to obtain high robustness with respect to a body motion of the subject 3. The possibility that continuous blood pressure measurement is interrupted due to a body motion of the subject 3 significantly decreases as compared with the known technology.

Although the first embodiment illustrates an example in which second-order differentiation is performed as the differential calculation process, the diastolic phase Td and the notch phase Tn may be detected by performing first-order differentiation. It is possible to obtain high robustness with respect to a body motion of the subject 3 as compared with the known technology even when using first-order differentiation.

The difference Δt in pulse wave transit time is obtained from the difference between the peak time t1 of the second-order differential value of the first blood vessel diameter D1 and the peak time t2 of the second-order differential value of the second blood vessel diameter D2. The processing device 40 according to the first embodiment calculates the pulse wave velocity PWV from the difference Δt in pulse wave transit time and the probe-to-probe distance Lp. The processing device 40 calculates the blood pressure P based on the pulse wave velocity PWV using the blood pressure calculation equation according to the first embodiment. Note that the peak times t1 and t2 are times (timings) that correspond to the diastolic phase Td. The difference Δt in pulse wave transit time may be obtained from the difference between peak times t3 and t4 that correspond to the notch phase Tn, and the pulse wave velocity PWV may be calculated from the difference Δt in pulse wave transit time and the probe-to-probe distance Lp.

The blood pressure calculation equation according to the first embodiment is described below.

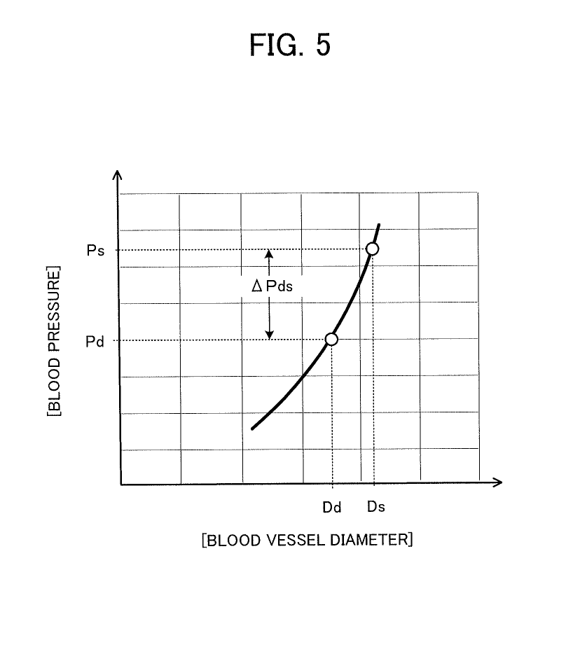

It is known that the blood vessel diameter-blood pressure characteristics in an unpressurized state are non-linear as illustrated in FIG. 5, for example. The measured blood pressure P, the systolic blood pressure Ps, the diastolic blood pressure Pd, the measured blood vessel diameter D, the systolic blood vessel diameter Ds, the diastolic blood vessel diameter Dd, and the stiffness parameter β have the relationship represented by the equations (1) and (2) (see above).

The relationship between the pulse wave velocity PWV and the elasticity of a blood vessel is represented by the following equation (3) (Moens-Korteweg equation). Note that h is the wall thickness of the blood vessel, r is the radius of the blood vessel, ρ is the blood density, and Einc is the incremental elastic modulus. The incremental elastic modulus Einc is represented by the following equation (4).

$$\begin{matrix}PWV = \sqrt{\frac{Einc \cdot h}{2 r ρ}} & ( 3 ) \\ Einc = \frac{Δ Pr \text{/} h}{Δ r \text{/} r} = \frac{Δ P \cdot {r}^{2}}{h Δ r} = \frac{Δ P \cdot {D}^{2}}{2 h Δ D} & ( 4 )\end{matrix}$$

Substituting the equation (4) into the equation (3), and transforming the resulting equations yield the following equation (5).

$$PWV = \sqrt{\frac{D \cdot Δ P}{2 ρ \cdot Δ D}} \qquad ( 5 )$$

Differentiating the equation (1) using the measured blood vessel diameter D, and transforming the resulting equation yield the following equation (6).

$$\frac{Δ P}{Δ D} = \frac{β}{Dd} \cdot P ( D ) \qquad ( 6 )$$

Substituting the equation (6) into the equation (5) yields the following equation (7), and transforming the equation (7) yields the following equation (8) (i.e., the blood pressure calculation equation according to the first embodiment). The blood pressure calculation equation according to the first embodiment is an equation that represents the relationship between the stiffness parameter β, the blood vessel diameter D, and the pulse wave velocity PWV.

$$\begin{matrix}PWV = \sqrt{\frac{D \cdot β}{2 ρ \cdot Dd} \cdot P ( D )} & ( 7 ) \\ P ( D , PWV ) = \frac{2 ρ}{β} \cdot \frac{Dd}{D} \cdot {PWV}^{2} & ( 8 )\end{matrix}$$

Since a change in the blood density ρ (see the equation (8)) is very small, the blood density ρ can be regarded as a constant. The stiffness parameter β is a constant that can be calibrated using the equation (2) (prior to the start of the measurement) from the calibration diastolic blood pressure Pd0 and the calibration systolic blood pressure Ps0 measured by the calibration blood pressure measurement unit 20, and the calibration diastolic blood vessel diameter Dd0 and the calibration systolic blood vessel diameter Ds0 measured by the ultrasonic measurement control unit 30 during the calibration period.

Therefore, it is necessary to measure the pulse wave velocity PWV and the blood vessel diameter D in order to implement continuous blood pressure measurement on a beat basis.

The pulse wave velocity PWV and the blood vessel diameter D substituted into the equation (8) have a specific relationship. Specifically, the pulse wave velocity PWV is calculated by calculating the time difference of arrival between the first blood vessel diameter D1 and the second blood vessel diameter D2 that correspond to the diastolic phase Td or the notch phase Tn as the difference Δt in pulse wave transit time. The blood vessel diameter D substituted into the equation (8) is the blood vessel diameter that corresponds to the diastolic phase Td or the notch phase Tn used when calculating the difference Δt in pulse wave transit time. The diastolic blood pressure Pd and the notch blood pressure Pn are thus calculated using the equation (8).

It is known that the systolic blood pressure Ps, the diastolic blood pressure Pd, and the notch blood pressure Pn have a specific relationship. Therefore, the systolic blood pressure Ps is calculated from the diastolic blood pressure Pd and the notch blood pressure Pn calculated using the equation (8).

According to the first embodiment, continuous blood pressure measurement on a beat basis is implemented in this manner.

According to the known technology that calculates blood pressure from a blood vessel diameter based on the equation (1), the blood vessel diameter D serves as an exponent. Therefore, when an error is mixed into the measured blood vessel diameter D due to a body motion of the subject 3, the blood pressure to be calculated is significantly affected. On the other hand, the equation (8) according to the first embodiment does not use the blood vessel diameter D as an exponent, and the blood vessel diameter D is not raised. Therefore, the effect of a measurement error in the blood vessel diameter D on the blood pressure to be calculated is significantly small as compared with the known technology. This makes it possible to improve robustness with respect to a body motion of the subject 3.

Functional Configuration

A functional configuration that implements the first embodiment is described below.

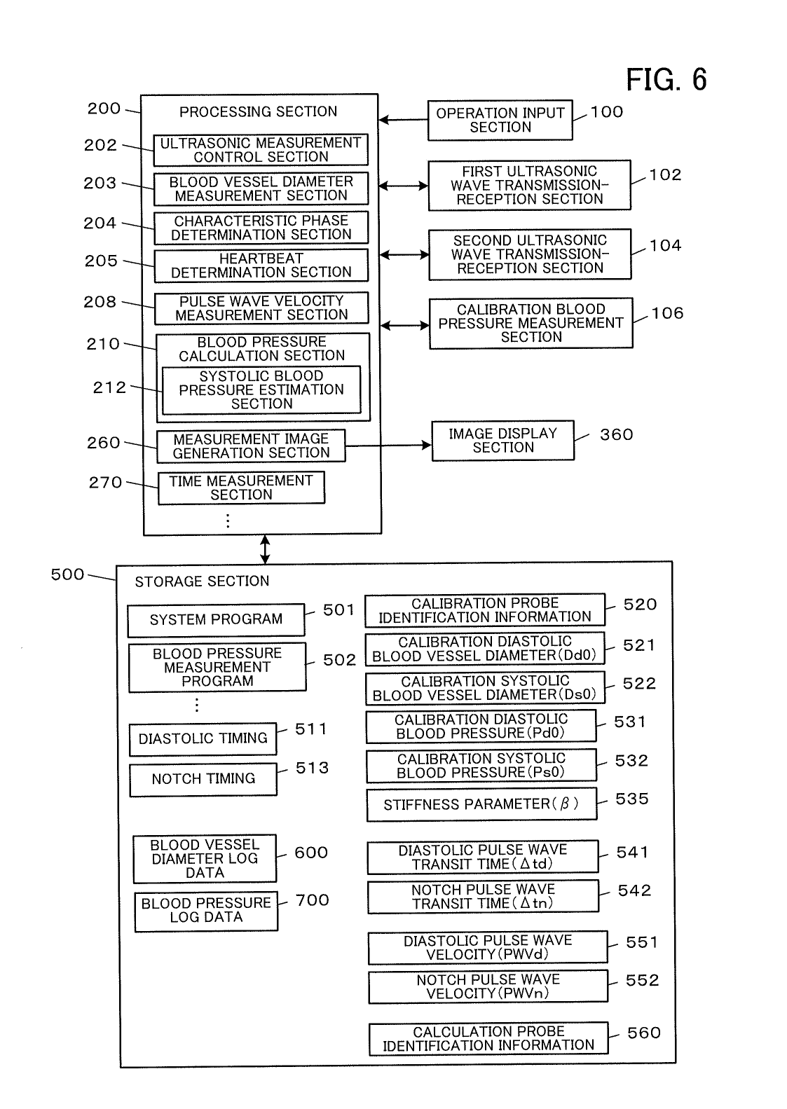

FIG. 6 is a block diagram illustrating a functional configuration example of the blood pressure measurement device 10 according to the first embodiment. The blood pressure measurement device 10 includes an operation input section 100, a first ultrasonic wave transmission-reception section 102, a second ultrasonic wave transmission-reception section 104, a calibration blood pressure measurement section 106, a processing section 200, an image display section 360, and a storage section 500.

The operation input section 100 receives an operation input performed by the operator, and outputs an operation input signal that corresponds to the operation input to the processing section 200. The operation input section 100 may be implemented by a button switch, a lever switch, a dial switch, a trackpad, a mouse, a touch panel, or the like. The touch panel 12 and the keyboard 14 illustrated in FIG. 1 correspond to the operation input section 100.

The first ultrasonic wave transmission-reception section 102 and the second ultrasonic wave transmission-reception section 104 transmit (apply) an ultrasonic wave used for the ultrasonic measurement, and receive the reflected wave based on a transmission control signal output from the processing section 200. For example, the first ultrasonic wave transmission-reception section 102 and the second ultrasonic wave transmission-reception section 104 are implemented by an ultrasonic vibration device or a driver circuit that drives an ultrasonic vibration device. The first ultrasonic probe 31 and the second ultrasonic probe 32 that are provided to the ultrasonic measurement control unit 30 illustrated in FIG. 1 correspond to the first ultrasonic wave transmission-reception section 102 and the second ultrasonic wave transmission-reception section 104, respectively.

The calibration blood pressure measurement section 106 is a means that acquires blood pressure that is used as a calibration standard. The calibration blood pressure measurement section 106 outputs the measured blood pressure information to the processing section 200. The calibration blood pressure measurement unit 20 illustrated in FIG. 1 corresponds to the calibration blood pressure measurement section 106.

The processing section 200 controls the entire blood pressure measurement device 10, and performs various calculation processes that calculate (measure) biological information about the subject 3. The processing section 200 is implemented by an electronic part such as a microprocessor (e.g., CPU or GPU), an application-specific integrated circuit (ASIC), a field-programmable gate array (FPGA), or an IC memory, for example. The processing section 200 exchanges data with (controls data exchange with) each functional section, and performs various calculation processes based on a given program, data, the operation input signal from the operation input section 100, and the like to calculate the biological information (blood pressure in the first embodiment) about the subject 3.

The processing section 200 includes an ultrasonic measurement control section 202, a blood vessel diameter measurement section 203, a characteristic phase determination section 204, a heartbeat determination section 205, a pulse wave velocity measurement section 208, a blood pressure calculation section 210, a measurement image generation section 260, and a time measurement section 270.

The ultrasonic measurement control section 202 controls the ultrasonic measurement. More specifically, the ultrasonic measurement control section 202 controls the transmission and the reception of an ultrasonic wave by the first ultrasonic wave transmission-reception section 102 and the second ultrasonic wave transmission-reception section 104, and performs a process that amplifies the reception signal that represents the reflected wave, and converts the reception signal into a digital signal, for example. The control device 33 provided to the ultrasonic measurement control unit 30 illustrated in FIG. 1 corresponds to the ultrasonic measurement control section 202.

The blood vessel diameter measurement section 203 continuously measures the diameter (blood vessel diameter) of the blood vessel 5 (e.g., carotid artery) based on the ultrasonic wave reception signal. A waveform that represents a temporal change in blood vessel diameter is obtained by the continuous measurement. In the first embodiment, the blood vessel diameter measurement section 203 measures the first blood vessel diameter D1 from the reception signal received by the first ultrasonic wave transmission-reception section 102, and measures the second blood vessel diameter D2 from the reception signal received by the second ultrasonic wave transmission-reception section 104. The front wall 5f and the rear wall 5r of the blood vessel 5 are detected from the reception signal (see FIG. 3), and the distance from the front wall 5f to the rear wall 5r is calculated when measuring the blood vessel diameter. Note that the blood vessel diameter may be measured using another method.

The characteristic phase determination section 204 determines the diastolic phase and the notch phase based on the waveform that represents a temporal change in blood vessel diameter that has been measured by the blood vessel diameter measurement section 203. The characteristic phase determination section 204 determines the diastolic phase and the notch phase that correspond to the first blood vessel diameter D1, and the diastolic phase and the notch phase that correspond to the second blood vessel diameter D2. The blood vessel diameter measurement section 203 determines the blood vessel diameter that corresponds to each characteristic phase determined by the characteristic phase determination section 204. More specifically, the blood vessel diameter measurement section 203 determines the first blood vessel diameter D1 that corresponds to the diastolic phase, and the first blood vessel diameter D1 that corresponds to the notch phase. The blood vessel diameter measurement section 203 also determines the second blood vessel diameter D2 that corresponds to the diastolic phase, and the second blood vessel diameter D2 that corresponds to the notch phase.

The characteristic phase determination section 204 performs a given differential calculation process on the waveform that represents a temporal change in blood vessel diameter to determine the diastolic phase and the notch phase (i.e., characteristic phases) of the pulse wave. In the first embodiment, the characteristic phase determination section 204 performs a second-order differentiation process, and detects the timing at which the second-order differential value satisfies the peak condition (i.e., a condition whereby the second-order differential value is equal to or larger than the reference value) to determine each characteristic phase.

The heartbeat determination section 205 determines the range of heartbeat within the ultrasonic measurement results from the determination results of the characteristic phase determination section 204. The heartbeat determination section 205 may have a function of calculating the heart rate.

The pulse wave velocity measurement section 208 measures the pulse wave velocity PWV through the blood vessel 5. In the first embodiment, the pulse wave velocity measurement section 208 calculates the difference Δt in pulse wave transit time corresponding to the diastolic phase Td and the notch phase Tn, and calculates the pulse wave velocity PWV from the difference Δt and the probe-to-probe distance Lp. Specifically, the pulse wave velocity measurement section 208 calculates the diastolic pulse wave velocity PWVd and the notch pulse wave velocity PWVn.

The blood pressure calculation section 210 calculates the blood pressure by performing a given calculation process that uses the blood vessel diameter D measured by the blood vessel diameter measurement section 203 and the pulse wave velocity PWV as variables. In the first embodiment, the blood pressure calculation section 210 calculates the blood pressure by performing the calculation process using the equation (8) in which the blood pressure is proportional to the second power of the pulse wave velocity, and is proportional to the reciprocal of the blood vessel diameter D. In other words, the blood pressure calculation section 210 calculates the blood pressure by performing the calculation process in which the proportional constant is specified based on the index value (stiffness parameter β) that represents the stiffness of the blood vessel 5 (that has been set during the calibration process), and the calibration diastolic blood vessel diameter Dd0 of the blood vessel 5 (see the equation (8)).

The blood pressure calculation section 210 includes a systolic blood pressure estimation section 212. The blood pressure calculation section 210 performs a calculation process that uses the diastolic blood vessel diameter Dd and the diastolic pulse wave velocity PWVd to calculate the diastolic blood pressure Pd, and performs a calculation process that uses the notch blood vessel diameter Dn and the notch pulse wave velocity PWVn to calculate the notch blood pressure Pn. The systolic blood pressure estimation section 212 performs a given systolic blood pressure estimation-calculation process that uses the diastolic blood pressure Pd and the notch blood pressure Pn to calculate the systolic blood pressure Ps. More specifically, the systolic blood pressure estimation section 212 calculates the systolic blood pressure Ps using the following equation (9) on the assumption that the notch blood pressure Pn is the mean arterial pressure (average arterial pressure).

Ps=3·Pn−2·Pd (9)

The measurement image generation section 260 generates various operation screens (images) that are necessary for the blood pressure measurement, and an image that displays the measurement results, and outputs the generated images to the image display section 360. The image display section 360 displays the image data output from the measurement image generation section 260. The touch panel 12 illustrated in FIG. 1 corresponds to the image display section 360.

The time measurement section 270 measures the measurement time. The time measurement method may be appropriately selected. For example, a system clock signal may be used.

The storage section 500 is implemented by a storage medium (e.g., IC memory, hard disk, or optical disk). The storage section 500 stores various programs and various types of data (e.g., data used during the calculation process performed by the processing section 200). The storage medium 43 provided to the control board 41 included in the processing device 40 illustrated in FIG. 1 corresponds to the storage section 500. Note that the processing section 200 and the storage section 500 need not necessarily be connected through an internal bus circuit included in the device. The processing section 200 and the storage section 500 may be connected through a communication line (e.g., local area network (LAN) or Internet). In this case, the storage section 500 may be implemented by an external storage device that is provided separately from the blood pressure measurement device 10.

The storage section 500 stores a system program 501, a blood pressure measurement program 502, a diastolic timing 511, and a notch timing 513. The storage section 500 also stores calibration probe identification information 520, a calibration diastolic blood vessel diameter 521, a calibration systolic blood vessel diameter 522, a calibration diastolic blood pressure 531, a calibration systolic blood pressure 532, a stiffness parameter 535, a diastolic pulse wave transit time 541, a notch pulse wave transit time 542, a diastolic pulse wave velocity 551, and a notch pulse wave velocity 552. The storage section 500 further stores blood vessel diameter log data 600 and blood pressure log data 700. Note that the storage section 500 may appropriately store additional information such as various determination flags, a time measurement counter value, and the like.

The system program 501 causes the blood pressure measurement device 10 to implement a basic input-output function as a computer. The processing section 200 executes the system program 501, and executes the blood pressure measurement program 502 to implement the functions of the ultrasonic measurement control section 202, the blood vessel diameter measurement section 203, the characteristic phase determination section 204, the heartbeat determination section 205, the pulse wave velocity measurement section 208, the blood pressure calculation section 210, the measurement image generation section 260, the time measurement section 270, and the like. Note that some of these functional sections may be implemented by hardware such as an electronic circuit.

The diastolic timing 511 and the notch timing 513 stored in the storage section 500 include information about the latest heartbeat timing (i.e., time information that represents each characteristic phase). The time information is information about a measurement time 601 included in the blood vessel diameter log data 600. The diastolic timing 511 and the notch timing 513 include time information about the first blood vessel diameter D1 and time information about the second blood vessel diameter D2.

The calibration probe identification information 520 includes information that represents whether the blood vessel diameter measured using the first ultrasonic probe 31 or the blood vessel diameter measured using the second ultrasonic probe 32 was used for the calibration process.

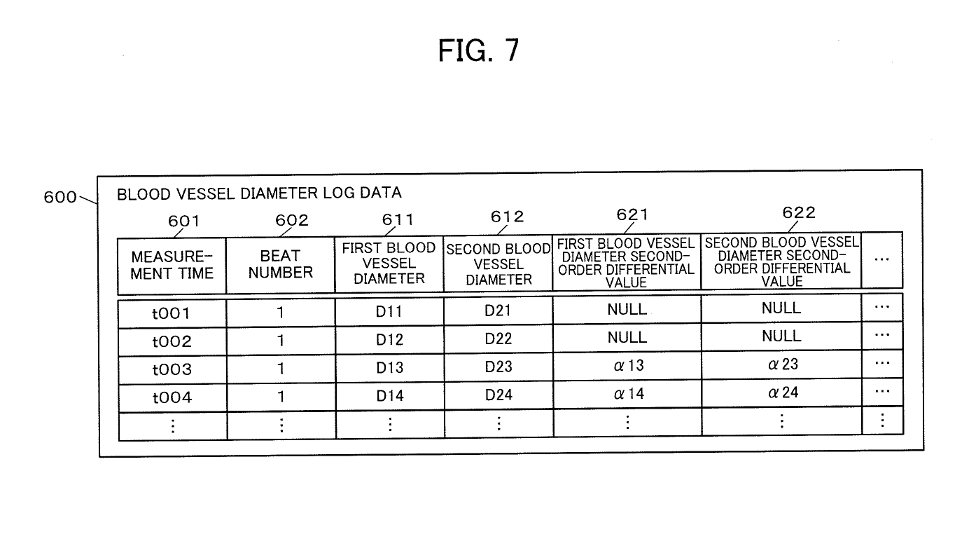

The blood vessel diameter log data 600 includes time-series information about the blood vessel diameter during the measurement. As illustrated in FIG. 7, the blood vessel diameter log data 600 includes the measurement time 601 that corresponds to each ultrasonic measurement cycle, a beat number 602 that represents the beat at the measurement time (e.g., a value that represents the number of each beat counted from the start of the measurement), a first blood vessel diameter 611 measured at the measurement time, a second blood vessel diameter 612 measured at the measurement time, a first blood vessel diameter second-order differential value 621, and a second blood vessel diameter second-order differential value 622 in a linked manner, for example. Note that the blood vessel diameter log data 600 may appropriately include additional data. In FIG. 7, the beat number 602 is identical (“1”) in spite of the passing of the measurement time 601 (“t001”, “t002”, “t003”, and “t004”) (i.e., the data illustrated in FIG. 7 corresponds to an identical beat). A waveform that represents a temporal change in blood vessel diameter is obtained by extracting the first blood vessel diameter 611 and the second blood vessel diameter 612 in time series. The first blood vessel diameter second-order differential value 621 and the second blood vessel diameter second-order differential value 622 are set to “NULL” at the times “t001” and “t002” since no data is available prior to the time “t001” (i.e., data necessary for calculating a second-order differential value has not been obtained).

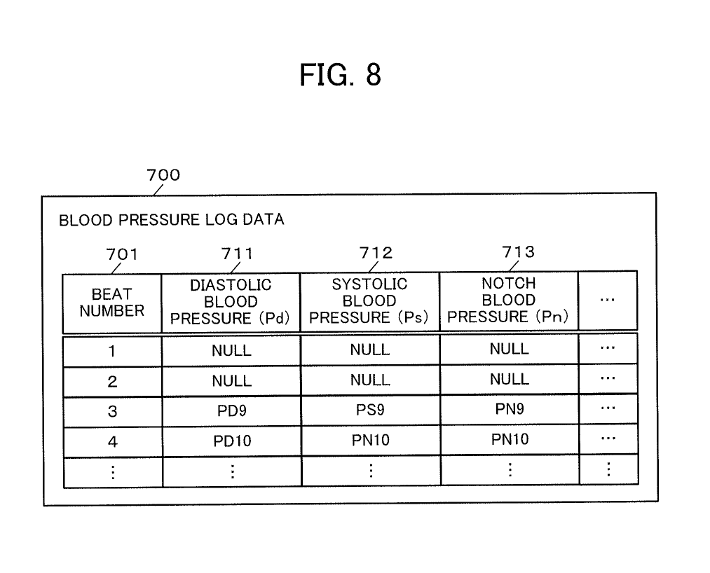

The blood pressure log data 700 includes the results of continuous blood pressure measurement on a beat basis. The blood pressure log data 700 includes time-series information about various blood pressures measured during the measurement. As illustrated in FIG. 8, the blood pressure log data 700 includes a beat number 701, a diastolic blood pressure 711, a systolic blood pressure 712, and a notch blood pressure 713 in a linked manner, for example. Note that the blood pressure log data 700 may appropriately include additional data.

Flow of Process

The operation of the blood pressure measurement device 10 is described below.

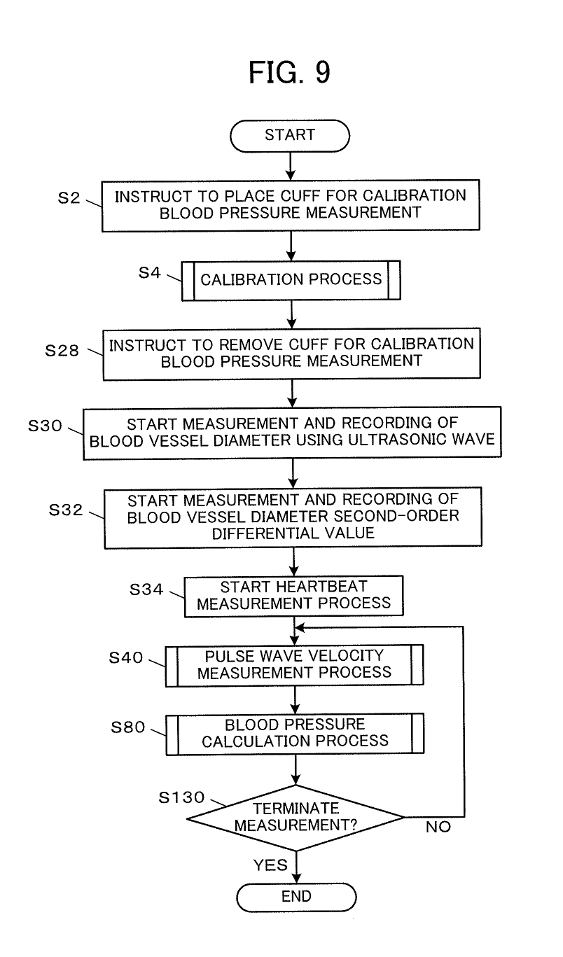

FIG. 9 is a flowchart illustrating the flow of the main process performed by the blood pressure measurement device 10 according to the first embodiment. Note that the first ultrasonic probe 31 and the second ultrasonic probe 32 are attached to the subject 3 in advance.

The blood pressure measurement device 10 displays an instruction that instructs the operator to place the cuff 21 of the calibration blood pressure measurement unit 20 around the upper arm of the subject 3 on the touch panel 12 (step S2). The display screen includes a placement completion operation input icon. When an operation input performed on the icon has been detected, the blood pressure measurement device 10 performs the calibration process (step S4).

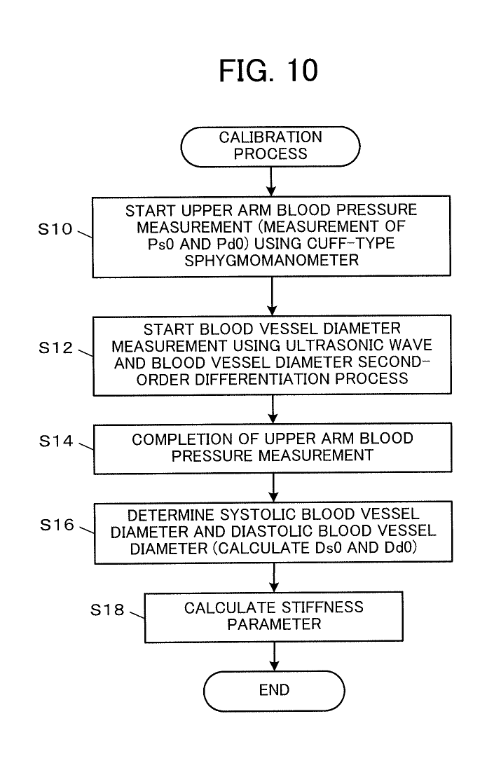

FIG. 10 is a flowchart illustrating the flow of the calibration process according to the first embodiment. As illustrated in FIG. 10, the blood pressure measurement device 10 starts a calibration upper arm blood pressure measurement process (step S10).

About several tens of seconds is required to complete the calibration blood pressure measurement process. The blood pressure measurement device 10 starts the process that measures the diameter of the blood vessel 5 using the first ultrasonic probe 31 and the second ultrasonic probe 32, and the blood vessel diameter second-order differentiation process during the calibration blood pressure measurement process (step S12). The measurement results are stored as the blood vessel diameter log data 600 (see FIG. 7).

When the calibration blood pressure measurement process has been completed, the calibration diastolic blood pressure Pd0 and the calibration systolic blood pressure Ps0 are transmitted from the calibration blood pressure measurement unit 20 to the processing device 40, and stored in the storage section 500 (step S14) (see FIG. 6).

The blood pressure measurement device 10 determines the diastolic phase and the systolic phase from the change in the first blood vessel diameter D1 and the change in the second blood vessel diameter D2 stored as the blood vessel diameter log data 600 during the calibration measurement process performed by the calibration blood pressure measurement unit 20, and determines the calibration diastolic blood vessel diameter Dd0 and the calibration systolic blood vessel diameter Ds0 (step S16). For example, the blood pressure measurement device 10 performs a statistical process (e.g., mean value calculation process or median value selection process) on the diastolic blood vessel diameter determined during the calibration measurement process (period) to determine the calibration diastolic blood vessel diameter Dd0. Likewise, the blood pressure measurement device 10 performs a statistical process (e.g., mean value calculation process or median value selection process) on the systolic blood vessel diameter determined during the calibration measurement process (period) to determine the calibration systolic blood vessel diameter Ds0.

Note that a change in blood vessel diameter becomes a maximum (i.e., the measurement accuracy increases) when the diameter of the blood vessel 5 is measured. Therefore, the change in the first blood vessel diameter D1 and the change in the second blood vessel diameter D2 may be compared, and the calibration diastolic blood vessel diameter Dd0 and the calibration systolic blood vessel diameter Ds0 may be determined from the first blood vessel diameter D1 or the second blood vessel diameter D2, whichever is larger with respect to the change.

The blood pressure measurement device 10 calculates the stiffness parameter β according to the equation (2) using the calibration diastolic blood pressure Pd0, the calibration systolic blood vessel diameter Ds0, the calibration diastolic blood vessel diameter Dd0, and the calibration systolic blood vessel diameter Ds0 (step S18). The proportional constant (=(2ρ/β)·Dd0) in the equation (8) (blood pressure calculation equation) is thus determined (i.e., the calibration process has been completed).

Again referring to FIG. 9, the blood pressure measurement device 10 displays an instruction that instructs the operator to remove the cuff 21 of the calibration blood pressure measurement unit 20 from the upper arm of the subject 3 on the touch panel 12 (step S28).

The display screen includes a removal completion operation input icon. When an operation input performed on the icon has been detected, the blood pressure measurement device 10 starts the blood pressure measurement process. More specifically, the blood pressure measurement device 10 clears the blood vessel diameter log data 600, starts the measurement and the recording of the first blood vessel diameter D1 and the second blood vessel diameter D2 (step S30), and starts the calculation and the recording of the first blood vessel diameter second-order differential value and the second blood vessel diameter second-order differential value (step S32). The blood pressure measurement device 10 then starts the heartbeat determination process using the heartbeat determination section 205 (step S34). The blood pressure measurement device 10 repeats the pulse wave velocity measurement process using the pulse wave velocity measurement section 208 (step S40) and the blood pressure calculation process using the blood pressure calculation section 310 (step S80) on a beat basis.

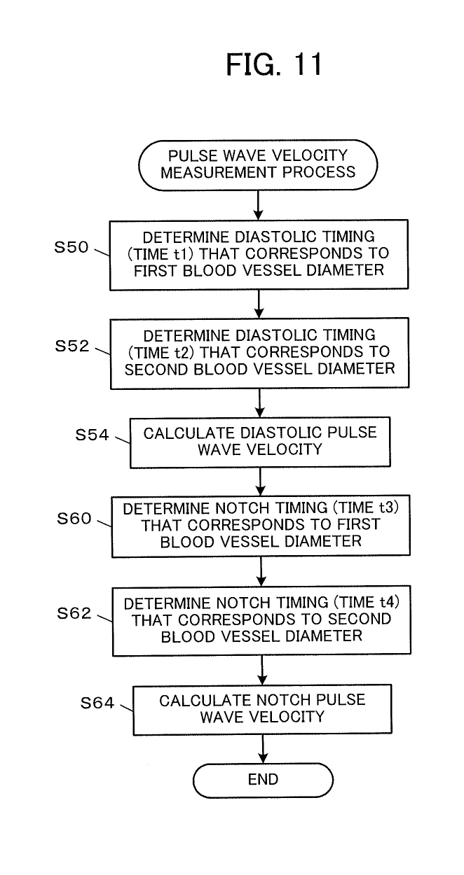

FIG. 11 is a flowchart illustrating the flow of the pulse wave velocity measurement process according to the first embodiment.

The blood pressure measurement device 10 determines the diastolic timing 511 that corresponds to the first blood vessel diameter D1 and the diastolic timing 511 that corresponds to the second blood vessel diameter D2 based on the blood vessel diameter log data 600 (steps S50 and S52). The blood pressure measurement device 10 calculates the difference between these diastolic timings (i.e., diastolic pulse wave transit time Δtd), and calculates the diastolic pulse wave velocity PWVd from the diastolic pulse wave transit time Δtd and the probe-to-probe distance Lp that is known in advance (step S54).

The blood pressure measurement device 10 then determines the notch timing 513 that corresponds to the first blood vessel diameter D1 and the notch timing 513 that corresponds to the second blood vessel diameter D2 based on the blood vessel diameter log data 600 (steps S60 and S62). The blood pressure measurement device 10 calculates the difference between these notch timings (i.e., notch pulse wave transit time Δtn), and calculates the notch pulse wave velocity PWVn from the notch pulse wave transit time Δtn and the probe-to-probe distance Lp that is known in advance (step S64). The blood pressure measurement device 10 then terminates the pulse wave velocity measurement process.

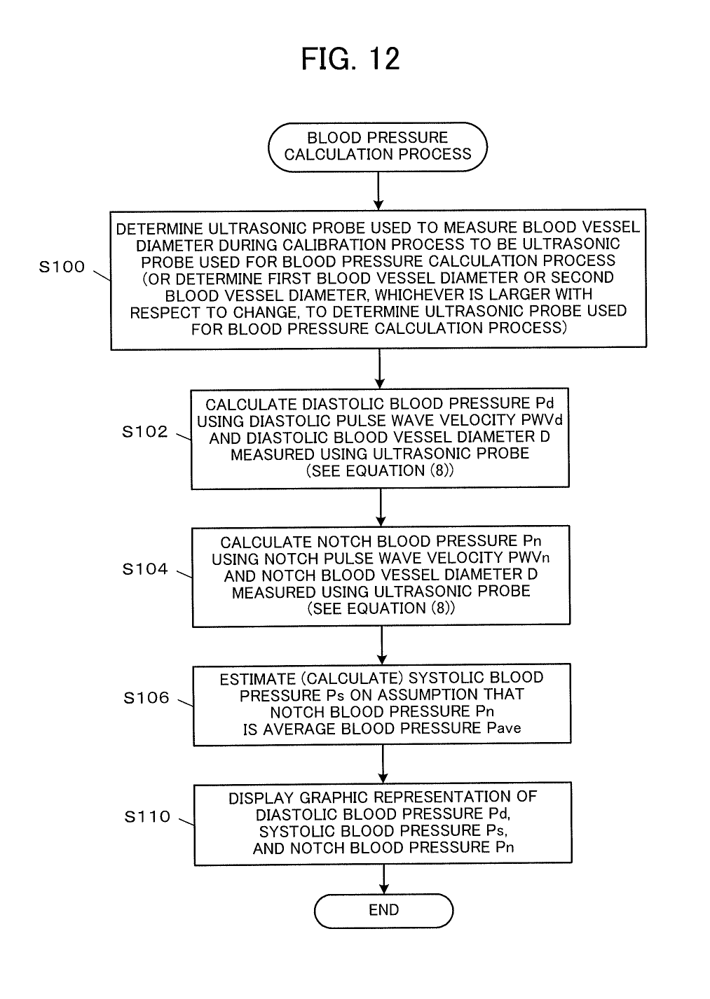

FIG. 12 is a flowchart illustrating the flow of the blood pressure calculation process according to the first embodiment. The blood pressure measurement device 10 performs the blood pressure calculation process using the ultrasonic probe used to measure the blood vessel diameter during the calibration process. Alternatively, the blood pressure measurement device 10 calculates the change in the first blood vessel diameter 611 and the change in the second blood vessel diameter 612 that correspond to the latest beat from the blood vessel diameter log data 600, and determines the first blood vessel diameter 611 or the second blood vessel diameter 612, whichever is larger with respect to the change, to determine the ultrasonic probe used for the blood pressure calculation process (step S100).

The blood pressure measurement device 10 calculates the blood pressure P using the diastolic pulse wave velocity PWVd and the blood vessel diameter D at the diastolic timing 511 that corresponds to the ultrasonic probe (see the equation (8)), and stores the calculated blood pressure P as the blood pressure log data 700 (i.e., diastolic blood pressure Pd) (step S102).

The blood pressure measurement device 10 calculates the blood pressure P using the notch pulse wave velocity PWVn and the blood vessel diameter D at the notch timing 513 that corresponds to the ultrasonic probe (see the equation (8)), and stores the calculated blood pressure P as the blood pressure log data 700 (i.e., notch blood pressure Pn) (step S104).

The blood pressure measurement device 10 estimates (calculates) the systolic blood pressure Ps using the equation (9) on the assumption that the notch blood pressure Pn is the average blood pressure Pave, and stores the estimated (calculated) systolic blood pressure Ps as the blood pressure log data 700 (step S106).

The blood pressure measurement device 10 then displays the diastolic blood pressure Pd, the systolic blood pressure Ps, and the notch blood pressure Pn on the touch panel 12 (step S110). In this case, it is preferable to also display information about the heart rate, the blood vessel diameter, and the like together with the diastolic blood pressure Pd, the systolic blood pressure Ps, and the notch blood pressure Pn. Note that the notch blood pressure Pn need not necessarily be displayed.

Again referring to FIG. 9, the blood pressure measurement device 10 determines whether or not the measurement termination condition has been satisfied (step S130). In the first embodiment, the blood pressure measurement device 10 determines that the measurement termination condition has been satisfied when a given measurement termination operation input has been performed using the touch panel 12 or the keyboard 14. A timer may be started at the start of the measurement, and the blood pressure measurement device 10 may determine that the measurement termination condition has been satisfied when a given time has elapsed. When the blood pressure measurement device 10 has determined that the measurement termination condition has been satisfied, the blood pressure measurement device 10 terminates the process. When the blood pressure measurement device 10 has determined that the measurement termination condition has not been satisfied, the blood pressure measurement device 10 performs the steps S40 and S80 again on a beat basis.

Validation of Effects

Data measured using the known method (known technology) that calculates blood pressure from a blood vessel diameter using the equation (1) was compared with data measured using the method according to the first embodiment that calculates blood pressure using the equation (8).

The following experimental conditions were used.

a) Blood pressure was measured using a tonometry-type sphygmomanometer while measuring the diameter (blood vessel diameter) of the carotid artery of a human subject using an ultrasonic wave.
b) The subject was prompted to change the position of the neck, and the blood vessel diameter, the blood pressure, the pulse wave velocity, the stiffness parameter, and the like were measured in the initial position and the post-change position that causes a measurement error in blood vessel diameter.
c) Various values including the stiffness parameter β that require calibration were calibrated in the initial position.

The stiffness parameter β was calculated from each value measured in the post-change position as a value that objectively represents the difference between the initial position and the post-change position. It was found that the stiffness parameter β changed by “0.46” from the value in the initial position (during calibration). The difference in pulse wave velocity between the initial position and the post-change position was about 18 cm/s.

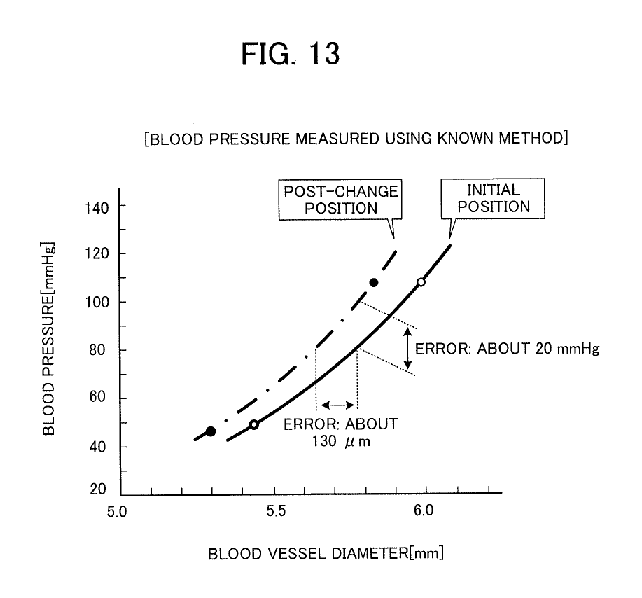

FIG. 13 is a graph illustrating the relationship between the blood vessel diameter and the blood pressure when the experiments were performed using the known method. The curves illustrated in FIG. 13 were estimated from the measured blood vessel diameter and blood pressure. The curve that corresponds to the initial position is indicated by the solid line, and the curve that corresponds to the post-change position is indicated by the dash-dotted line. When the blood vessel diameter was about 5.78 mm, the blood pressure measured in the initial position was 80 mmHg, and the blood pressure measured in the post-change position was 100 mmHg. Specifically, an error of about 20 mmHg occurred.

Note that an error in blood vessel diameter was about 130 μm when calculated at a blood pressure of about 80 mmHg.

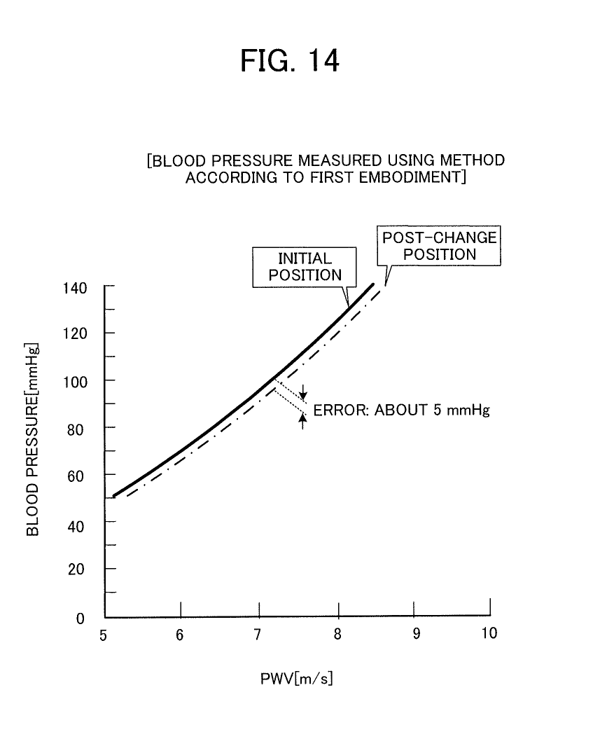

FIG. 14 is a graph illustrating the relationship between the pulse wave velocity PWV and the blood pressure when the experiments were performed using the method according to the first embodiment. The curve that corresponds to the initial position is indicated by the solid line, and the curve corresponds to the post-change position is indicated by the dash-dotted line. The blood pressure measured in the post-change position at the pulse wave velocity corresponding to a blood pressure of 100 mmHg in the initial position was 95 mmHg. Specifically, the error was as small as about 5 mmHg.

As described above, the first embodiment implements blood pressure measurement that exhibits excellent robustness with respect to a change in position as compared with the known method, and makes it possible to implement high-accuracy continuous blood pressure measurement for a long time.

Note that the embodiments to which the invention can be applied are not limited to the first embodiment. Various modifications may be appropriately made, such as adding other elements, omitting some of the elements, or changing some of the elements.

First Modification

Although the first embodiment has been described above taking an example in which the calibration blood pressure measurement process is performed prior to the start of the measurement, the calibration blood pressure measurement process may be omitted. In this case, the relationship between a physical characteristic parameter (e.g., age, sex, height, and weight) of the subject 3 and the stiffness parameter β is determined in advance using a statistical method, and stored in the storage section 500 as table data. Alternatively, a function that derives the stiffness parameter β using the physical characteristic parameter (e.g., age, sex, height, and weight) of the subject 3 is set, and stored in the storage section 500. For example, a function that derives the stiffness parameter β from the age of the subject 3 is represented by the following equation (10).

β=A·[age]+B (10)

Note that A and B are constants. The constant A is selected from the range from 0.05 to 0.3, and the constant B is selected from the range from 2 to 5.

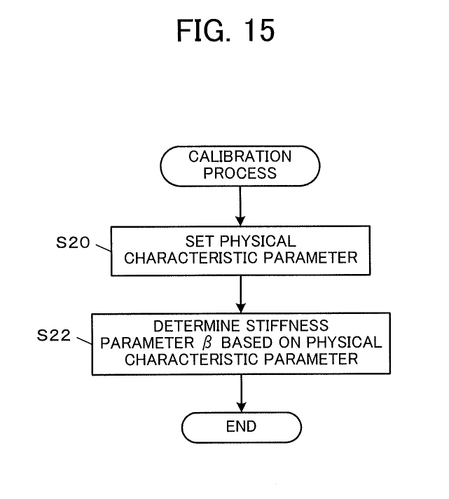

When implementing the calibration process (see FIG. 15), the physical characteristic parameter of the subject is set (input) (step S20), and the stiffness parameter β is determined from the set physical characteristic parameter referring to the table data, or determined using the function that derives the stiffness parameter β (step S22).

Second Modification

When the calibration blood pressure measurement unit 20 can measure the notch blood pressure in addition to the diastolic blood pressure and the systolic blood pressure, the notch blood pressure and the notch blood vessel diameter may be used instead of the diastolic blood pressure and the diastolic blood vessel diameter when calculating the stiffness parameter β using the equation (2).

Third Modification

Although the first embodiment has been described above taking an example in which the ultrasonic probe used to calculate blood pressure is determined (selected) taking account of a change in blood vessel diameter due to pulsation (step S100) (see FIG. 12), the configuration is not limited thereto. For example, the ultrasonic probe that corresponds to the first blood vessel diameter D1 or the second blood vessel diameter D2, whichever is closer (in the diastolic phase and the systolic phase) to the calibration diastolic blood vessel diameter 521 and the calibration systolic blood vessel diameter 522 (see FIG. 6) may be used. When the absolute value of the difference in blood pressure calculated from the first blood vessel diameter D1 and the second blood vessel diameter D2 is larger than a given value, it may be determined that it is impossible to implement the measurement, and a message may be output using light or sound.

Fourth Modification

The method that estimates (calculates) the systolic blood pressure Ps in the step S106 of the blood pressure calculation process (see FIG. 12) is not limited to the method described above in connection with the first embodiment.

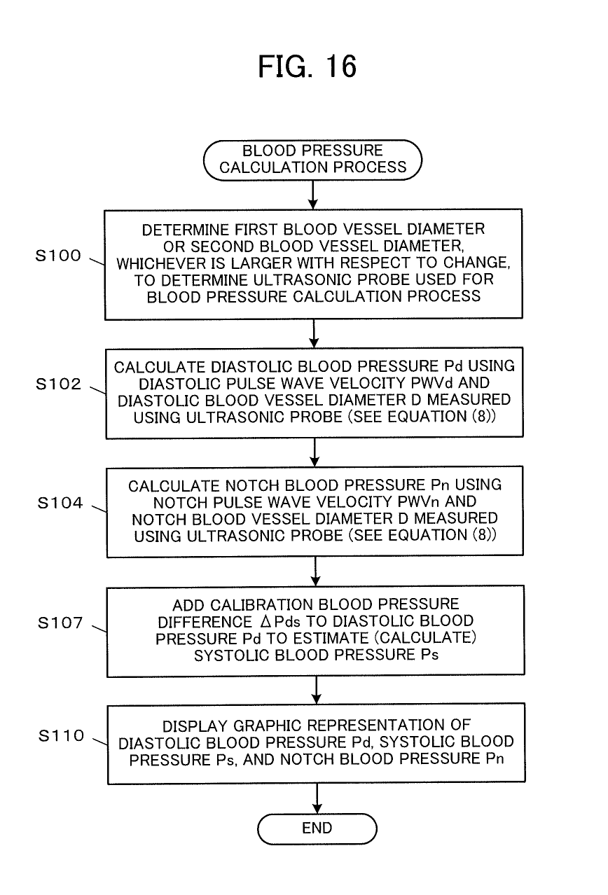

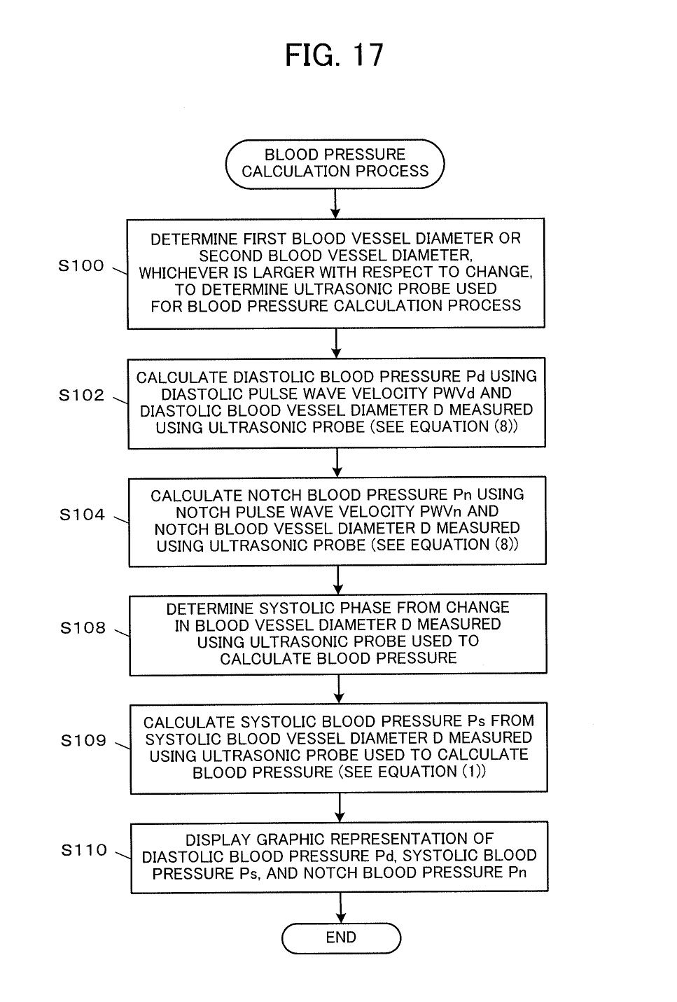

As illustrated in FIG. 16, the calibration blood pressure difference ΔPds (see FIG. 5) may be calculated from the calibration diastolic blood pressure 531 and the calibration systolic blood pressure 532 (see FIG. 6), and added to the diastolic blood pressure Pd calculated in the step S102 to estimate (calculate) the systolic blood pressure Ps (step S107) instead of performing the step S106, for example. As illustrated in FIG. 17, the systolic phase may be determined from a change in the blood vessel diameter that corresponds to the ultrasonic probe used to calculate blood pressure (step S108), and the systolic blood pressure Ps may be calculated using the β method that uses the equation (1) (step S109) instead of performing the step S106.

Fifth Modification

Although the first embodiment has been described above taking an example in which the systolic blood pressure Ps is estimated (calculated), the estimation (calculation) of the systolic blood pressure Ps may be omitted. Specifically, only the diastolic blood pressure Pd, or the diastolic blood pressure Pd and the notch blood pressure Pn may be displayed (output).

The entire disclosure of Japanese Patent Application No. 2015-105496 filed on May 25, 2015 is expressly incorporated by reference herein.

Although only some embodiments of the present invention have been described in detail above, those skilled in the art will readily appreciate that many modifications are possible in the embodiments without materially departing from the novel teachings and advantages of this invention. Accordingly, all such modifications are intended to be included within scope of this invention.

What is claimed is:

1. A blood pressure measurement device comprising:
a blood vessel diameter measurement section that measures a blood vessel diameter of an artery;
a pulse wave velocity measurement section that measures a pulse wave velocity through the artery; and
a blood pressure calculation section that performs a given calculation process that uses the blood vessel diameter and the pulse wave velocity as variables to calculate blood pressure.

2. The blood pressure measurement device as defined in claim 1,
the blood pressure calculation section calculating the blood pressure by performing the calculation process in which the blood pressure is proportional to a second power of the pulse wave velocity, and is proportional to a reciprocal of the blood vessel diameter.

3. The blood pressure measurement device as defined in claim 1,
the blood vessel diameter measurement section and the pulse wave velocity measurement section performing measurement with respect to an identical characteristic phase of a pulse wave.

4. The blood pressure measurement device as defined in claim 1, further comprising:
a characteristic phase determination section that performs a given differential calculation process on a waveform that represents a temporal change in the blood vessel diameter to determine a diastolic phase and a notch phase of a pulse wave.

5. The blood pressure measurement device as defined in claim 1,
the blood vessel diameter measurement section measuring a diastolic blood vessel diameter and a notch blood vessel diameter,
the pulse wave velocity measurement section measuring a diastolic pulse wave velocity and a notch pulse wave velocity, and
the blood pressure calculation section calculating diastolic blood pressure by performing the calculation process that uses the diastolic blood vessel diameter and the diastolic pulse wave velocity, calculating notch blood pressure by performing the calculation process that uses the notch blood vessel diameter and the notch pulse wave velocity, and calculating systolic blood pressure by performing a given systolic blood pressure estimation-calculation process that uses the diastolic blood pressure and the notch blood pressure.

6. The blood pressure measurement device as defined in claim 5,
the blood pressure calculation section calculating the systolic blood pressure by performing the systolic blood pressure estimation-calculation process on an assumption that the notch blood pressure is an average arterial pressure.

7. The blood pressure measurement device as defined in claim 1, further comprising:
ultrasonic probes that transmit and receive an ultrasonic wave to and from the artery, the ultrasonic probes including a first ultrasonic probe that is in charge of an upstream side of the artery, and a second ultrasonic probe that is in charge of a downstream side of the artery,
the blood vessel diameter measurement section measuring the blood vessel diameter based on a reception signal received by the first ultrasonic probe or a reception signal received by the second ultrasonic probe, and
the pulse wave velocity measurement section measuring the pulse wave velocity based on the reception signal received by the first ultrasonic probe and the reception signal received by the second ultrasonic probe.

8. The blood pressure measurement device as defined in claim 7,
the first ultrasonic probe and the second ultrasonic probe transmitting and receiving the ultrasonic wave to and from the carotid artery, subclavian artery, or aorta.

9. The blood pressure measurement device as defined in claim 8,
the blood vessel diameter measurement section measuring the blood vessel diameter based on the reception signal received by the first ultrasonic probe and the blood vessel diameter based on the reception signal received by the second ultrasonic probe, and
the blood pressure calculation section using the blood vessel diameter based on the reception signal received by the first ultrasonic probe or the blood vessel diameter based on the reception signal received by the second ultrasonic probe, whichever is larger with respect to a change due to pulsation.

10. The blood pressure measurement device as defined in claim 7,
the first ultrasonic probe and the second ultrasonic probe being thin probes that are attached to a skin surface of a subject.

11. A blood pressure measurement method comprising:
measuring a blood vessel diameter of an artery;
measuring a pulse wave velocity through the artery; and
performing a given calculation process that uses the blood vessel diameter and the pulse wave velocity as variables to calculate blood pressure.
